(19) United States
(12) Patent Application Publication
(10) Pub. No.: US 2025/0387604 A1
(43) Pub. Date: Dec. 25, 2025

(54) PORTABLE SELF SANITIZING MICRONEEDLE DEVICE

(71) Applicant: Daisy Jing, Pasadena, CA (US)

(72) Inventor: Daisy Jing, Pasadena, CA (US)

(21) Appl. No.: 19/304,455

(22) Filed: Aug. 19, 2025

Related U.S. Application Data
(63) Continuation of application No. 16/656,187, filed on Oct. 17, 2019, now abandoned.

(51) Int. Cl. A61M 37/00; A61M 5/00; A61M 5/158

(52) U.S. Cl. CPC A61M 37/0015; A61M 5/001; A61M 5/158; A61M 2005/1586; A61M 2205/11

(57) ABSTRACT

A portable, self-sanitizing microneedle device similar in size and shape to a pen, or marker that can be held and manipulated by a human hand. The microneedle device is comprised of a main body with a handle attached to a circular base support structure, a second circular platform structure having screw threads to connect with screw threads on the inside of a substantially conical-shaped cap with a hexagonal base that holds a disinfecting solution, and a third circular platform structure comprising the microneedle support structure covered with a substrate holding a plurality of microneedles. The cap is sealingly and removably attached to the base support structure over the microneedle array using a plurality of sealing surfaces within the cap and on the support structures to create a tight seal that prevents the leakage of the disinfecting solution, and keeps the microneedles sanitized for use at all times, even when traveling.

### SEQUENCE LISTING OR PROGRAM

Not Applicable

### TECHNICAL FIELD OF THE INVENTION

This invention relates in general to microneedle devices. More particularly, the invention relates to a portable self-sanitizing microneedle device.

### BACKGROUND OF THE INVENTION

Skin rejuvenation with topical applications of commercially available creams and lotions to reduce the appearance of age-related wrinkles is a well-known practice especially among women. Exfoliation of the outer epidermal layer of the skin through chemical peels and skin re-surfacing using microderm abrasion devices are also well known in the art. The transdermal delivery of therapeutic fluids into the deeper layers of the skin requires the penetration of the epidermal stratum corneum of the skin which forms a barrier, preventing the entry of environmental agents into the skin. A microneedle device accomplishes the goal of penetrating the skin's stratum corneum to deliver transdermal fluids deeper into the skin for cosmetic rejuvenation of the skin as well as to heal and repair skin subject to injury, or scarring.

A microneedle device in general is comprised of a plurality of tiny needles intended to penetrate and perforate the skin's outer layers to facilitate the delivery of cosmetic, or therapeutic fluids into the inner layers of the skin. Examples of prior art devices using microneedles for skin treatment are described in U.S. Pat. No. 9,492,647 to Stumber, U.S. Pat. No. 9,358,376 to Altarac, and U.S. Pat. No. 7,131,960 to Trautman. U.S. Pat. No. 9,492,647 to Stumber describes a microneedle array applicator comprising a planar apparatus holding the microneedle array and a drive mechanism that results in the needles composed of silicon, or a biodegradable polymer laden with an active ingredient in the region of the tip, to break off and get deposited in the skin. U.S. Pat. No. 9,358,376 to Altarac describes a microneedle roller infusion system with the needles enclosed in a housing with a disposable cartridge holding the active agent. U.S. Pat. No. 7,131,960 to Trautman describes an applicator with a plurality of stratum corneum-piercing micro protrusions to form micro slits through which an agent can be delivered to the skin.

The popularity of microneedle devices for cosmetic enhancement, rejuvenation, or treatment of damaged skin has also led to the side effects of bacterial skin infections brought about through the use of un-sanitized needles in the devices. A lay person using the microneedle device at home is not trained in the proper procedure to disinfect and sanitize the needles before use. Therefore, they are compelled to seek the costly services of an experienced aesthetician, or dermatologist to treat their skin with a microneedle device for cosmetic, or therapeutic purposes. Accordingly, there is a need in the art for a microneedle device that provides a means to easily and effectively disinfect and sanitize the microneedles in the device by a user within the confines of their home, or when traveling, to prevent bacterial infections to the skin, during treatment with the microneedles. There is also a need in the art for a microneedle device that is compact and light in weight to be portable without the microneedle disinfecting solution contained within the device leaking out into the surrounding areas during transport. The present invention provides such a microneedle device.

### SUMMARY OF THE INVENTION

The present invention is a portable, self-sanitizing microneedle device that overcomes the deficiencies in the prior art for such a microneedle device. The microneedle device of the invention can be safely used within the confines of one's home without the help of a professional aesthetician, or dermatologist.

It is an object of the present invention to provide a compact and portable microneedle device that effectively self-sanitizes the microneedles for use when on the go, or while traveling.

Yet another object of the present invention is to provide a microneedle device that provides a tight seal between the cap holding the microneedle sanitizing solutions and the base structure of the device holding the microneedles, to prevent leakage of the solutions when the device is in use at home, or when the device is transported during travels.

In the exemplary embodiment of the present invention of a microneedle device, the device is comprised of a main body that is similar in size and shape to a pen, or marker that can be held and manipulated by a human hand. In other embodiments, the size of the device may be larger than a pen, or marker, yet capable of being manipulated by one hand. In embodiments of the device, the main body of the device has a handle with a top broad end and a tapered distal end. In some embodiments the handle may have a different shape to the tapered distal end. The top broad end of the handle is connected to a circular broad base support structure with two additional tiers of circular platform structures decreasing in circumference from the base support structure to the top third platform structure. In this embodiment of the device, the third platform structure which forms the microneedle array support structure is covered with a layer of substantially planar substrate material holding a plurality of microneedles with their pointed and sharp ends projecting upward from the substrate.

In the exemplary embodiment of the microneedle device of the present invention, the base support structure and the microneedle array support structure holding the microneedles on the substrate material are covered with a substantially conical-shaped cap having a hexagonal base and an internal cavity to contain the disinfecting solutions to sanitize the microneedles after each use. The cap of the microneedle device of the present invention is structured to form a tight seal over the base structure of the device holding the microneedles to prevent leakage of the disinfecting solution held within the cap over the microneedles, while they are being sanitized.

In the exemplary embodiment of the microneedle device of the invention, the cap covering the microneedle array has a substantially circular mouth with a rim that is sealingly and removably coupled to the base structure of the device. The cap is contemplated to sealingly couple with the body of the microneedle device in at least three ways, 1) a first sealing surface comprising the rim at the mouth end of the cap and a contact point at the bottom end of the base support structure, 2) a second sealing surface comprising the inside of the cap and a second contact point on the base structure just above the first contact point on the base support structure in a substantially perpendicular direction to the first sealing surface, and 3) a third sealing surface comprising a screw thread on the inside of the cap engaging with a screw thread on the second platform structure above the base support structure. In this embodiment of the microneedle device, a crevice formed just below the top platform structure holding the microneedle array is adapted to contain an air bubble to allow for a tight seal between the cap and the base support structure to prevent the microneedle disinfecting solutions contained in the cap from leaking out into the surroundings. In all embodiments of the device, the cap has an internal cavity capable of holding a measure of disinfecting solution that can interact with the microneedles to effectively disinfect and sanitize the needles after each use.

In embodiments of the microneedle device, the cap has a flat planar bottom which acts as a stand to support the device to be held up on a surface.

In yet another embodiment of the microneedle device of the present invention, a battery-powered motor is enclosed within the handle of the device to vibrate the microneedles and agitate the disinfecting solution in the cap, to facilitate an enhanced and thorough cleaning and sanitizing of the microneedles.

The features and advantages of the present invention described in this summary of the invention will become obvious and appreciated by one skilled in the art when viewed in conjunction with the accompanying drawings, detailed description of the invention, and the appended claims.

### BRIEF DESCRIPTION OF THE DRAWINGS

The accompanying drawings, which are incorporated herein and form a part of the specification, illustrate the present invention and, together with the description, further serve to explain the principles of the invention and to enable a person skilled in the pertinent art to make and use the invention.

FIG. 1 is a perspective view of the microneedle device of the present invention showing the cap of the device separated from the body of the device.

FIG. 2 is a perspective view of the microneedle device of the present invention showing the cap attached to the body of the device.

FIG. 3 is a perspective view of the microneedle device of the present invention illustrating the base of the cap acting as a stand to hold up the device on a surface.

FIG. 4 is a perspective view of the base structure of the microneedle device connected to the handle, highlighting the plurality of sealing conduits that enable a leak-proof seal between the base structure and the cap of the device.

FIG. 5 is a cross sectional view of the microneedle device of the present invention illustrating the position of the cap over the plurality of the sealing conduits.

FIG. 6 is an enlarged illustration showing the position of the air bubble generating crevice below the microneedle holding platform structure in relation to the three sealing surfaces.

### DETAILED DESCRIPTION OF THE INVENTION

The present invention is a microneedle device that is equipped with a cap that is capable of forming a tight seal over the base of the device holding the microneedle array such that the disinfecting solution held within the cap to clean and sanitize the microneedles, does not leak from the device even when the device is transported. In the exemplary embodiment of the microneedle device of the present invention, the device is similar in size and shape to a pen, or marker that can be held and manipulated by a human hand. In some embodiments, the device may be larger than the size of a pen, or marker, yet capable of being held and manipulated by a hand to carry out the treatment of the skin with the device.

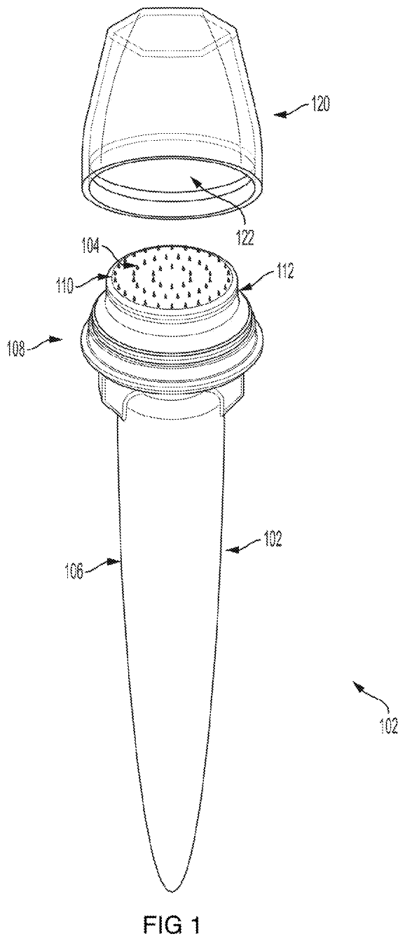

Referring now to the drawings wherein like numerals represent like components in the several views presented and discussed, and more particularly referring now to FIG. 1 the figure is a perspective view of the exemplary embodiment of the microneedle device 102 of the present invention. The main body of the microneedle device 102 is comprised of a handle 106, a base support structure 108 that connects the handle 106 to the microneedle support structure 110. In this embodiment of the microneedle device 102 the microneedle support structure 110 is covered with a substantially planar substrate that holds a plurality of microneedles 104 with their broad base ends embedded within the substrate and their sharp ends projecting upwards from the substrate. The diameter of the microneedles 104 is contemplated to be greater at their base ends and tapered to a sharp point at their distal ends. In the preferred embodiment of the microneedle device 102 the microneedles 104 are arranged in concentric circles on the substantially planar substrate covering the microneedle support structure 110. In some embodiments of the device, the microneedles 104 may be arranged vertically, horizontally, or in other patterns on the substrate. In some embodiments, the microneedles 104 are fabricated to hold a hollow shaft in the center of the microneedle to form a conduit through which fluid can enter and exit.

The microneedle device 102 further comprises a substantially conical shaped cap 120 with a hexagonal base that is sealably and removably coupled to the base support structure 108 to cover the microneedles 104 held on the planar substrate on the microneedle support structure 110. The cap 120 is configured with an internal cavity 122 that is capable of holding a measured volume of liquid preferably a disinfectant solution to clean and disinfect the microneedles 104 after each use. In some embodiments of the microneedle device 102, the cap 120 has a level mark displayed to fill with a measure of the disinfecting solution.

The cap 120 sealingly and removably couples with the main body of the microneedle device 102 in a plurality of ways to form a tight, leak-proof seal. The rim of the cap 120 engages with a first sealing surface on the base support structure 108. The inside of the cap 120 just above the mouth of the cap 120 engages with a second sealing surface on the base support structure, and a third sealing surface comprises a screw thread on the inside surface of the cap contacting a matching screw thread on the second platform structure just above the base support structure 108. A crevice 112 formed just below the microneedle support structure 110 is adapted to hold an air bubble which allows for a tight seal between the mouth of the cap 120 and the base support structure 108 to prevent the leakage of disinfecting solutions held inside the cap 120.

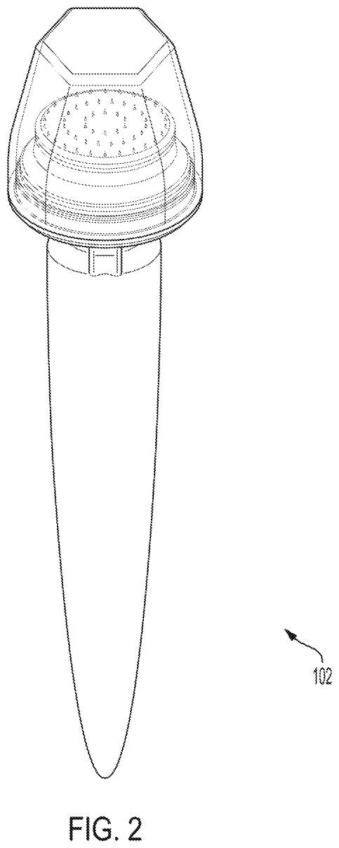

Referring again to the drawings, FIG. 2 is a perspective view of the microneedle device of the present invention showing the manner in which the cap of the device is attached to the body of the device covering the microneedle array. The circular mouth of the cap engages with the base support structure through the rim of the cap and the inside of the cap just above the mouth of the cap, and a screw thread on the inside surface of the cap engages with a matching screw thread on the second platform structure just above the base support structure to form a tight seal in such a manner that prevents the disinfecting solutions held inside the cap from leaking out.

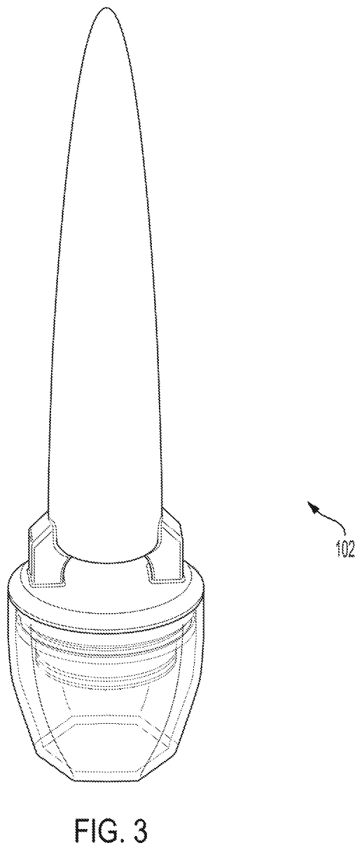

FIG. 3 is a perspective view of the microneedle device of the present invention illustrating the base of the cap acting as a stand to hold up the device on a surface. This feature is especially helpful in filling and emptying the cap with the disinfecting solutions. When filling the cap with the solutions, the cap can be unscrewed from the base of the main body of the device, filled with the solution to the level mark indicated on the cap and then the main body holding the microneedles can be screwed back on to the cap while the cap is resting on its base. Similarly, when emptying the disinfecting solution from the cap after the needles are sanitized, the main body of the device can be held up by the base of the cap while being unscrewed from the cap to empty the solution from the cap.

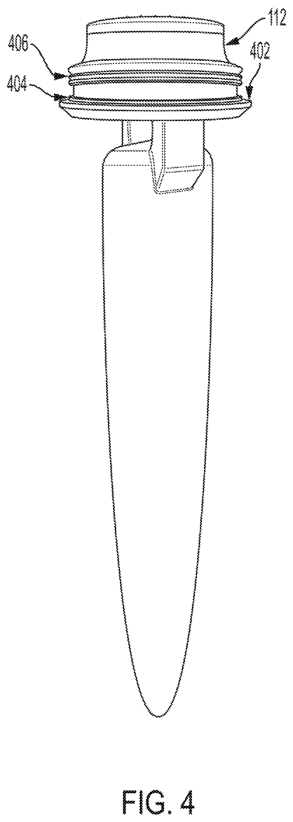

FIG. 4 is a perspective view of the base structure of the microneedle device connected to the handle, highlighting the plurality of sealing conduits that enable a leak-proof seal between the base structure and the cap of the device. The cap sealingly and removably engages with the structures connected to the handle of the device at least in three ways. The rim of the cap (not seen in this view) engages with a first sealing surface 402 on the base support structure. The inside of the cap just above the mouth of the cap engages with a second sealing surface 404 on the base support structure, and a third sealing surface 406 comprises a screw thread on the inside surface of the cap contacting a matching screw thread on the second platform structure just above the base support structure. In some embodiments, the first sealing surface 402 and the second sealing surface 404 may be at an angle. The third sealing surface 406 comprises a continuous low pitch thread. This thread fits in with the corresponding thread on the inside of the cap. The third sealing surface 406 with the screw thread having the continuous low pitch thread when combined with the first sealing surface 402 and the second sealing surface 404 prevents leakage of the disinfecting solution held within the device.

As would be known by one skilled in the art, a thread pitch is the distance between threads expressed in millimeters. The screw thread disclosed herein also has a low helix angle, which is the angle on which the threads are set to the axis of the microneedle device.

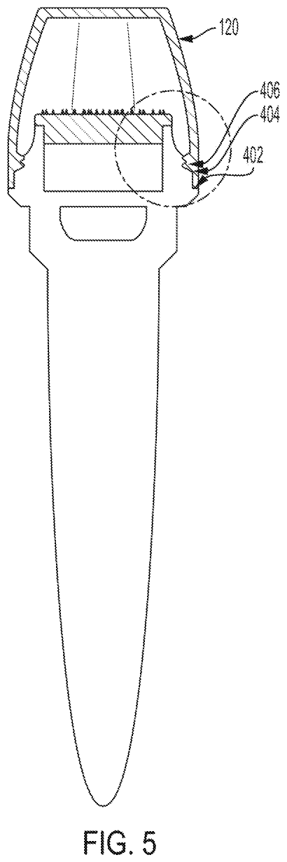

FIG. 5 is a cross sectional view of the microneedle device of the present invention illustrating the position of the cap over the plurality of the sealing conduits. The rim of the cap 120 engages with a first sealing surface 402 on the base support structure. The inside of the cap just above the mouth of the cap engages with a second sealing surface 404 on the base support structure, and a third sealing surface comprises a screw thread on the inside surface of the cap contacting a matching screw thread 406 on the second platform structure just above the base support structure. The tight sealing feature of the microneedle base and cap combination is an important feature of the present invention especially as it relates to the shape and structure of the cap of the device. For example, with a hexagonal shaped cap, it was found that the sealing with the microneedle base was not tight, and required the use of an O-ring or a gasket to generate a tight seal between the cap and the microneedle base. A tight seal was formed and no leakage was seen with the substantially conical-shaped cap with a circular mouth and rim without the need for an O ring in the present invention.

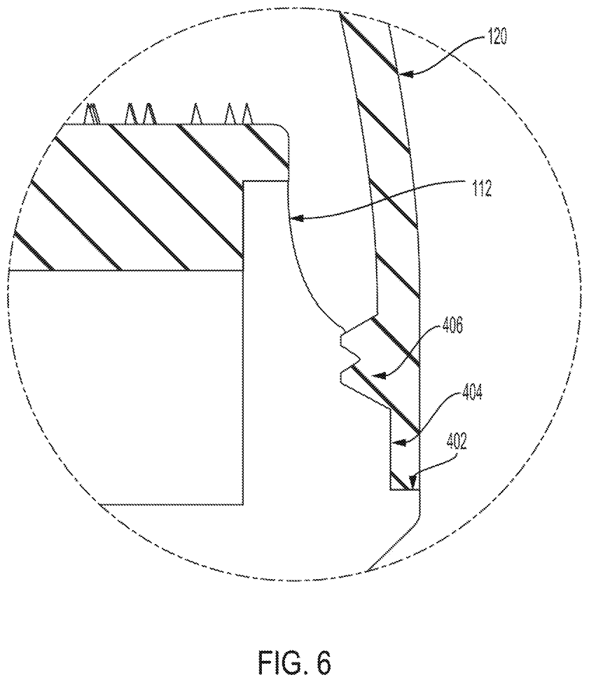

FIG. 6 is an enlarged illustration showing the position of the air bubble-generating crevice 112 below the microneedle holding platform structure in relation to the three sealing surfaces. More specifically, the figure shows the air bubble generating crevice 112 just below the microneedle support structure. By trial and error it was found that to achieve a tight seal between the microneedle support structure and the cap requires the presence of the crevice 112 just below the microneedle support structure. When the cap is filled with disinfecting solution, up to a line marked on the cap, the presence of the crevice in the device enables an air bubble to go in between so that the solution will not overflow, and the needles will remain submerged in the solution so that they are effectively sanitized. The air bubble can be seen when the cap is tilted upside down with the solution in the cap. The air bubble is intended to flow into the crevices of the threads on the sealing surface 406 so that it is not readily noticeable when held upright and the disinfecting solution perfectly hits the needles without leaking.

FIG. 6 also illustrates the positions of the third sealing surface 406 comprising screw threads for engaging with the screw threads on the inside of the cap and the second sealing surface 404 that engages with the inside of the cap just above the mouth of the cap and the first sealing surface 402 that engages with the rim on the mouth of the cap together creating a tight leak-proof seal between the cap holding the disinfecting solution and the base support structure.

In some embodiments, the cap is transparent (or at least partially transparent) to enable a user to see that cleaning solution is present in the cap, to see the level of the cleaning solution, and to see how dirty or clean the cleaning solution is. In general, one or more liquid level lines may be inscribed on the cap to help a user determine how much microneedle disinfecting solution should fill the cap. These lines which may be etched, or painted, and may comprise, a maximum and/or minimum depth line.

In some embodiments, the cap further comprises at least one reservoir member configured to maintain at least some of the disinfecting solution within the cavity of the cap even if the cap is removed from the needle base and rotated such that the cap's opening is positioned in a downward direction. For example, the cap may have a reservoir member comprising a sponge, foam, porous plastic, one or more baffles, that can help to maintain the disinfecting solution within the cavity of the cap even when the cap is not engaged with the microneedle base.

In use, once the microneedles protruding from the microneedle base support structure have soaked in the disinfecting solution, and are sanitized and cleaned, a user can remove the base structure from the cap, shake off any excess solution still remaining on the microneedles, and using the handle of the device, press the needles against his or her skin, forcing the plurality of microneedles to puncture the user's skin and allow any skin rejuvenating and/or treatment medicament applied to the skin, to penetrate the skin's stratum corneum. After use, the user can replace the cap filled with fresh disinfecting solution to clean and sanitize the microneedles till the next use. The time needed for disinfecting the microneedles before the next use depends on the type of disinfecting solutions used and how dirty the microneedles are after a use. Generally, with the right disinfecting solution, the microneedles would be disinfected within five minutes of soaking in the disinfecting solution.

In some embodiments, the microneedle device has a vibrating mechanism within the handle to cause the needles and/or the disinfecting solution to be vibrated and/or agitated during the cleaning process to enhance the cleaning process. For example, the handle of the microneedle device may comprise a battery that powers a motor, which causes vibration of the device and agitation of the disinfecting solution bathing over the microneedles.

In some embodiments, the substrate holding the microneedles comprises a compressible and/or deformable material that may provide comfort to the skin surface while using the device and/or allow the microneedle support structure to more easily conform to the contours of a person's skin as the device is being used. In some embodiments, the substrate holding the microneedles and/or the microneedle support structure may not comprise a compressible and/or deformable material.

The cap of the microneedle device is contemplated to be made from a material that is not compressible and/or deformable during regular use and atmospheric temperature and pressure. The cap is generally made of plastic, or other non-deformable material having a thickness that when engaged with the microneedle support structure device, prevents manual deformation.

While the present invention of a self-sanitizing portable microneedle device has thus been described through its preferred embodiments and related figures, it is to be understood that the embodiments of the present invention as described herein do not limit any application or scope of the invention and that the invention can be carried out and practiced in various ways and implemented in embodiments other than the ones outlined and described above. It should be understood and obvious to one skilled in the art that alternatives, modifications, and variations of the embodiments of the present invention may be construed as being within the spirit and scope of the appended claims.

1. A portable, self-sanitizing microneedle device comprising:
a main body structure;
said main body structure comprising, a substantially circular base support structure connected to a broad top end of a handle structure;
said handle structure having a tapered distal end;
wherein said circular base support structure is a first tier portion that comprises two additional tiers of circular platform tier structures extending from the base first tier portion up to a top, third tier portion decreasing in circumference from said base first tier portion through a second tier platform structure to a top third tier platform structure;
wherein said top third tier platform structure of the base support structure comprises a microneedle holding structure;
said microneedle holding structure covered with a substantially planar substrate;
wherein said substantially planar substrate comprises a plurality of microneedles;
said plurality of microneedles each having a broad base end embedded in said substrate and a tapered sharp end projecting upward from said substrate;
said plurality of microneedles arranged in concentric circles on said substrate;
wherein said second tier platform structure is located between said first tier portion of the base support structure and said top, third tier portion platform structure of the base support structure;
said second tier platform structure comprising a screw thread structure;
a crevice structure just below said microneedle holding structure to hold an air bubble;
a cap structure sealingly and removably coupled to said base structure; and
said cap structure having an internal cavity to hold a measure of disinfecting solution to disinfect and sanitize said plurality of microneedles.

2. The portable, self-sanitizing, microneedle device of claim 1 wherein the device is comprised of a main body that is similar in size and shape of a pen, or marker that can be held and manipulated by a human hand and easily transported.

3. The portable, self-sanitizing microneedle device of claim 1 wherein the device is larger than the size of a pen, or marker, yet capable of being held and manipulated by a hand to carry out the treatment of the skin with the device.

4. The portable, self-sanitizing micro needle device of claim 1 wherein the microneedles are arranged vertically, horizontally, or in other patterns on the substrate.

5. The portable, self-sanitizing, microneedle device of claim 1 wherein said cap structure is substantially conical-shaped with a flat hexagonal base.

6. The portable, self-sanitizing, microneedle device of claim 1 wherein said cap structure is sealingly and removably coupled to the base structure in at least three ways, 1) the rim of the cap engages with a first sealing surface on the base support structure, 2) the inside of the cap just above the mouth of the cap engages with a second sealing surface on the base support structure, and 3) a screw thread on the inside surface of the cap engages with a matching screw thread structure on the second platform structure just above the base support structure.

7. The portable, self-sanitizing microneedle device of claim 6 wherein the first sealing surface and the second sealing surface are at an angle.

8. The portable, self-sanitizing microneedle device of claim 6 wherein the third sealing surface comprises a continuous thread that engages with a matching screw thread on the inside of the cap.

9. The portable, self-sanitizing microneedle device of claim 6 wherein the engagement of the three sealing surfaces between the cap structure and the base structure of the device forms a tight seal to prevent leakage of the disinfecting solution held within the device.

10. The portable, self-sanitizing microneedle device of claim 1 wherein the air bubble within the crevice structure is generated when the cap is filled with a disinfecting solution, and the presence of the air bubble prevents the overflow and leakage of the solution, and facilitates the submerging of the microneedles in the disinfecting solution so that they are effectively sanitized.

11. The portable self-sanitizing, microneedle device of claim 1 wherein the cap is at least partially transparent to enable a user to see the level of the disinfecting solution within the cap, and to see how dirty or clean the solution is.

12. The portable, self-sanitizing, portable, microneedle device of claim 1 wherein the cap has one or more liquid level lines inscribed on the cap to help a user determine how much microneedle disinfecting solution should fill the cap, and wherein said level lines comprise, a maximum, or minimum depth line.

13. The portable, self-sanitizing, microneedle device of claim 1 wherein the flat base of the cap structure acts as a stand to hold up the microneedle device over a surface.

14. The portable, self-sanitizing, microneedle device of claim 1 wherein the cap further comprises at least one reservoir within the cap configured to maintain at least some of the microneedle disinfecting solution within the cavity of the cap even if the cap is removed from the base structure and rotated and the cap's opening is positioned in a downward direction.

15. The portable, self-sanitizing, microneedle device of claim 14 wherein the reservoir uses a sponge, foam, porous plastic, one or more baffles, that can help to maintain the disinfecting solution within the cavity of the cap even when the cap is not engaged with the microneedle base structure.

16. The portable, self-sanitizing, microneedle device of claim 1 wherein the cap structure of the microneedle device is made of a non-deformable material having a thickness that, when engaged with the microneedle support structure, prevents manual deformation during use.

17. The portable, self-sanitizing, microneedle device of claim 1 wherein the substrate holding the microneedles is of a deformable material that provides comfort to the skin surface while using the device and allows the microneedle support structure to more easily conform to the contours of a person's skin as the device is being used over the skin surface.

18. The portable, self-sanitizing, microneedle device of claim 1 wherein the substrate holding the microneedles is of a non-deformable material.

19. The microneedle device of claim 1 wherein the microneedle device has a vibrating mechanism comprising a battery powered motor within the handle structure to cause the needles and disinfecting solution to be agitated during the cleaning process to enhance the cleaning process.

20. The microneedle device of claim 1 wherein the microneedles are fabricated to hold a hollow shaft in the center of the microneedle to form a conduit through which fluid can enter and exit.
